SEQUENCE LISTING (38 sequences)

SEQ ID NO: 1 - DNA, 330 bp, Homo sapiens
  gene (1)..(330): proBDNF
gcccccatgaaagaagcaaacatccgaggacaaggtggcttggcctacccaggtgtgcggacccatgggactctggagagcgtgaatgggcccaaggcaggttcaagaggcttgacatcattggctgacactttcgaacacatgatagaagagctgttggatgaggaccagaaagttcggcccaatgaagaaaacaataaggacgcagacttgtacacgtccagggtgatgctcagtagtcaagtgcctttggagcctcctcttctctttctgctggaggaatacaaaaattacctagatgctgcaaacatgtccatgagggtccggcgc

SEQ ID NO: 2 - protein, 110 aa, Homo sapiens
  PEPTIDE (1)..(110): proBDNF precursor domain
APMKEANIRGQGGLAYPGVRTHGTLESVNGPKAGSRGLTSLADTFEHVIEELLDEDQKVRPNEENNKDADLYTSRVMLSSQVPLEPPLLFLLEEYKNYLDAANMSMRVRR

SEQ ID NO: 3 - protein, 5 aa, Artificial Sequence
  1D3 HCDR1
GYDMH

SEQ ID NO: 4 - protein, 16 aa, Artificial Sequence
  1D3 HCDR2
GLGMEGDSYYSASVKG

SEQ ID NO: 5 - protein, 7 aa, Artificial Sequence
  1D3 HCDR3
DVHGFDV

SEQ ID NO: 6 - protein, 16 aa, Artificial Sequence
  1D3 LCDR1
RSSQSLLYSNGYTYLD

SEQ ID NO: 7 - protein, 7 aa, Artificial Sequence
  1D3 LCDR2
MGSNRAS

SEQ ID NO: 8 - protein, 9 aa, Artificial Sequence
  1D3 LCDR3
MQALQTPLT

SEQ ID NO: 9 - protein, 115 aa, Artificial Sequence
  1D3 VH
QVQLVESGGGLIQPGGSMRLSCAASGFSLSGYDMHWVRQIAGKGLEWVAGLGMEGDSYYSASVKGRFTISRQDAKNSLYLEMKDLGGGDTAVYYCLRDVHGFDVWGQGTTVTVSS

SEQ ID NO: 10 - protein, 113 aa, Artificial Sequence
  1D3 VL
DVVMTQSPLSLPVTPGEPASISCRSSQSLLYSNGYTYLDWYLQRPGQSPQLLIYMGSNRASGVPDRFSGSGSGTDFTLKISRVEAEDVGVYYCMQALQTPLTFGGGTKLEIKR

SEQ ID NO: 11 - protein, 442 aa, Artificial Sequence
  heavy chain of 1D3
QVQLVESGGGLIQPGGSMRLSCAASGFSLSGYDMHWVRQIAGKGLEWVAGLGMEGDSYYSASVKGRFTISRQDAKNSLYLEMKDLGGGDTAVYYCLRDVHGFDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 12 - protein, 219 aa, Artificial Sequence
  light chain of 1D3
DVVMTQSPLSLPVTPGEPASISCRSSQSLLYSNGYTYLDWYLQRPGQSPQLLIYMGSNRASGVPDRFSGSGSGTDFTLKISRVEAEDVGVYYCMQALQTPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 5 aa, Artificial Sequence
  2H8 HCDR1
SYGMH

SEQ ID NO: 14 - protein, 17 aa, Artificial Sequence
  2H8 HCDR2
VISGSGDSTYYAESVKG

SEQ ID NO: 15 - protein, 10 aa, Artificial Sequence
  2H8 HCDR3
GILTGYVFDY

SEQ ID NO: 16 - protein, 16 aa, Artificial Sequence
  2H8 LCDR1
RSSQSLVSNDGNTYLN

SEQ ID NO: 17 - protein, 7 aa, Artificial Sequence
  2H8 LCDR2
MVSKWDS

SEQ ID NO: 18 - protein, 9 aa, Artificial Sequence
  2H8 LCDR3
MQSTHWPPT

SEQ ID NO: 19 - protein, 119 aa, Artificial Sequence
  2H8 VH
EVQLVETGGGLVQPGGSLRLSCAASGFTFSSYGMHWVRQAPGKGLEWVSVISGSGDSTYYAESVKGRFTISRDNARNTVYLQMNSLRAEDTAVYYCASGILTGYVFDYWGKGTMVTVSS

SEQ ID NO: 20 - protein, 113 aa, Artificial Sequence
  2H8 VL
DVVMTQSPLSLPVTLGQPASISCRSSQSLVSNDGNTYLNWFQQRPGQPPRRLIYMVSKWDSGVPDRFSGSGSGTDFTLRISRVEAEDVGVYYCMQSTHWPPTFGGGTKLEIKR

SEQ ID NO: 21 - protein, 446 aa, Artificial Sequence
  heavy chain of 2H8
EVQLVETGGGLVQPGGSLRLSCAASGFTFSSYGMHWVRQAPGKGLEWVSVISGSGDSTYYAESVKGRFTISRDNARNTVYLQMNSLRAEDTAVYYCASGILTGYVFDYWGKGTMVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSPGK

SEQ ID NO: 22 - protein, 219 aa, Artificial Sequence
  light chain of 2H8
DVVMTQSPLSLPVTLGQPASISCRSSQSLVSNDGNTYLNWFQQRPGQPPRRLIYMVSKWDSGVPDRFSGSGSGTDFTLRISRVEAEDVGVYYCMQSTHWPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 23 - protein, 243 aa, Artificial Sequence
  1D3scFv
QVQLVESGGGLIQPGGSMRLSCAASGFSLSGYDMHWVRQIAGKGLEWVAGLGMEGDSYYSASVKGRFTISRQDAKNSLYLEMKDLGGGDTAVYYCLRDVHGFDVWGQGTTVTVSSGGGGSGGGGSGGGGSDVVMTQSPLSLPVTPGEPASISCRSSQSLLYSNGYTYLDWYLQRPGQSPQLLIYMGSNRASGVPDRFSGSGSGTDFTLKISRVEAEDVGVYYCMQALQTPLTFGGGTKLEIKR

SEQ ID NO: 24 - DNA, 729 bp, Artificial Sequence
  1D3scFv
caggtgcagctggtggagtctgggggaggcctgatacagccgggggggtcgatgagactctcctgtgcagcctctggattcagcctcagtggatatgacatgcactgggtccgccaaattgcgggaaaaggtctggagtgggtcgccggtcttgggatggaaggtgactcatattattcagcctccgtgaagggccgattcaccatctccagacaagatgccaagaattccctgtatcttgaaatgaaggacctgggaggcggggacacggctgtctattactgtctaagagatgtccacggattcgacgtctggggccaagggaccacggtcaccgtctcgagtggtggaggcggttcaggcggaggtggttctggcggtggcggatcggatgttgtgatgactcagtctccactctccctgcccgtcacccctggagagccggcctccatctcctgcaggtctagtcagagcctcctgtatagtaatggatacacctatttggattggtacctgcagaggccagggcagtctccacagctcctgatctatatgggttctaatcgggcctccggggtccctgacaggttcagtggcagtggatcaggcacagattttacactgaaaatcagcagagtggaggctgaggatgttggggtttattactgcatgcaagctctacaaactcccctcactttcggcggagggaccaagctggagatcaaacgt

SEQ ID NO: 25 - protein, 247 aa, Artificial Sequence
  2H8 scFv
EVQLVETGGGLVQPGGSLRLSCAASGFTFSSYGMHWVRQAPGKGLEWVSVISGSGDSTYYAESVKGRFTISRDNARNTVYLQMNSLRAEDTAVYYCASGILTGYVFDYWGKGTMVTVSSGGGGSGGGGSGGGGSDVVMTQSPLSLPVTLGQPASISCRSSQSLVSNDGNTYLNWFQQRPGQPPRRLIYMVSKWDSGVPDRFSGSGSGTDFTLRISRVEAEDVGVYYCMQSTHWPPTFGGGTKLEIKR

SEQ ID NO: 26 - DNA, 741 bp, Artificial Sequence
  2H8 scFv
gaggtgcagctggtggagactgggggcggcttggtccagcctggggggtccctgagactctcctgtgcagcgtctggattcaccttcagtagctatggcatgcactgggtccgccaggctccagggaaggggctggagtgggtctcagttattagtggtagtggtgatagtacatactacgcagagtccgtgaagggccgcttcaccatctccagagacaatgccaggaacacggtgtatctgcaaatgaacagtctgagagccgaggacacggctgtatattattgtgcaagtggcattttgactggttatgtatttgactattggggcaaagggacaatggtcaccgtctcgagtggtggaggcggttcaggcggaggtggttctggcggtggcggatcggatgttgtgatgactcagtctccactctccctgcccgtcacccttggacagccggcctccatctcctgcaggtctagtcaaagcctcgtatccaatgatggaaacacctacttgaattggtttcagcagaggccaggccaacctccaaggcgcctaatttatatggtttctaagtgggactctggggtcccagacagattcagcggcagtgggtcaggcactgatttcacactgagaatcagcagggtggaggctgaggatgttggggtttattactgcatgcaaagtacacactggcctcccactttcggcggagggaccaagctggagatcaaacgt

SEQ ID NO: 27 - protein, 54 aa, Artificial Sequence
  E2
KVRPNEENNKDADLYTSRVMLSSQVPLEPPLLFLLEEYKNYLDAANMSMRVRRH

SEQ ID NO: 28 - protein, 36 aa, Artificial Sequence
  E5
VMLSSQVPLEPPLLFLLEEYKNYLDAANMSMRVRRH

SEQ ID NO: 29 - protein, 12 aa, Artificial Sequence
  DH
DAANMSMRVRRH

SEQ ID NO: 30 - protein, 56 aa, Artificial Sequence
  E1
PMKEANIRGQGGLAYPGVRTHGTLESVNGPKAGSRGLTSLADTFEHMIEELLDEDQ

SEQ ID NO: 31 - protein, 37 aa, Artificial Sequence
  E3
PMKEANIRGQGGLAYPGVRTHGTLESVNGPKAGSRGL

SEQ ID NO: 32 - protein, 37 aa, Artificial Sequence
  VP
TSLADTFEHMIEELLDEDQKVRPNEENNKDADLYTSR

SEQ ID NO: 33 - protein, 12 aa, Artificial Sequence
  VP
VMLSSQVPLEPP

SEQ ID NO: 34 - protein, 12 aa, Artificial Sequence
  LL
LLFLLEEYKNYL

SEQ ID NO: 35 - protein, 426 aa, Homo sapiens
  PEPTIDE (1)..(426): p75NTR
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSCKQNKQGASRPVNQTPPPEGEKLHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV

SEQ ID NO: 36 - protein, 831 aa, Homo sapiens
  PEPTIDE (1)..(831): sortilin
MERPWGAADGLSRWPHGLGLLLLLQLLPPSTLSQDRLDAPPPPAAPLPRWSGPIGVSWGLRAAAAGGAFPRGGRWRRSAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCKADLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSVCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQNSKSNSVPIILAIVGLMLVTVVAGVLIVKKYVCGGRFLVHRYSVLQQHAEANGVDGVDALDTASHTNKSGYHDDSDEDLLE

SEQ ID NO: 37 - protein, 1159 aa, Homo sapiens
  PEPTIDE (1)..(1159): SORCS2
MAHRGPSRASKGPGPTARAPSPGAPPPPRSPRSRPLLLLLLLLGACGAAGRSPEPGRLGPHAQLTRVPRSPPAGRAEPGGGEDRQARGTEPGAPGPSPGPAPGPGEDGAPAAGYRRWERAAPLAGVASRAQVSLISTSFVLKGDATHNQAMVHWTGENSSVILILTKYYHADMGKVLESSLWRSSDFGTSYTKLTLQPGVTTVIDNFYICPTNKRKVILVSSSLSDRDQSLFLSADEGATFQKQPIPFFVETLIFHPKEEDKVLAYTKESKLYVSSDLGKKWTLLQERVTKDHVFWSVSGVDADPDLVHVEAQDLGGDFRYVTCAIHNCSEKMLTAPFAGPIDHGSLTVQDDYIFFKATSANQTKYYVSYRRNEFVLMKLPKYALPKDLQIISTDESQVFVAVQEWYQMDTYNLYQSDPRGVRYALVLQDVRSSRQAEESVLIDILEVRGVKGVFLANQKIDGKVMTLITYNKGRDWDYLRPPSMDMNGKPTNCKPPDCHLHLHLRWADNPYVSGTVHTKDTAPGLIMGAGNLGSQLVEYKEEMYITSDCGHTWRQVFEEEHHILYLDHGGVIVAIKDTSIPLKILKFSVDEGLTWSTHNFTSTSVFVDGLLSEPGDETLVMTVFGHISFRSDWELVKVDFRPSFSRQCGEEDYSSWELSNLQGDRCIMGQQRSFRKRKSTSWCIKGRSFTSALTSRVCECRDSDFLCDYGFERSSSSESSTNKCSANFWFNPLSPPDDCALGQTYTSSLGYRKVVSNVCEGGVDMQQSQVQLQCPLTPPRGLQVSIQGEAVAVRPGEDVLFVVRQEQGDVLTTKYQVDLGDGFKAMYVNLTLTGEPIRHRYESPGIYRVSVRAENTAGHDEAVLFVQVNSPLQALYLEVVPVIGLNQEVNLTAVLLPLNPNLTVFYWWIGHSLQPLLSLDNSVTTRFSDTGDVRVTVQAACGNSVLQDSRVLRVLDQFQVMPLQFSKELDAYNPNTPEWREDVGLVVTRLLSKETSVPQELLVTVVKPGLPTLADLYVLLPPPRPTRKRSLSSDKRLAAIQQVLNAQKISFLLRGGVRVLVALRDTGTGAEQLGGGGGYWAVVVLFVIGLFAAGAFILYKFKRKRPGRTVYAQMHNEKEQEMTSPVSHSEDVQGAVQGNHSGVVLSINSREMHSYLVS

SEQ ID NO: 38 - protein, 678 aa, Homo sapiens
  PEPTIDE (1)..(678): Extracellular domain of p75NTR and sortilin
SAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCKADLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSVCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQNSKSN

(19) United States
(12) Patent Application Publication
(10) Pub. No.: US 2022/0064274 A1
(43) Pub. Date: Mar. 3, 2022

(54) USE OF PROBDNF REGULATOR IN B CELL-RELATED DISEASES

(71) Applicant: SHANGHAI YILE BIOTECHNOLOGY CO., LTD., Shanghai (CN)

(72) Inventors: Ruping DAI, Shanghai (CN); Xiumei CAI, Shanghai (CN); Huamao WANG, Shanghai (CN)

(21) Appl. No.: 17/041,559

(22) PCT Filed: Mar. 26, 2019
(86) PCT No.: PCT/CN2019/079735

§ 371 Date: Dec. 30, 2020

(30) Foreign Application Priority Data
Mar. 26, 2018 (CN) 201810254609.X

(51) Int. Cl. C07K 16/22; A61K 45/06; G01N 33/68

(52) U.S. Cl. CPC C07K 16/22; A61K 45/06; G01N 33/68; C07K 2317/76; C07K 2317/565; G01N 2333/4756; G01N 2800/52

(57) ABSTRACT

Disclosed is a use of a proBDNF regulator in B cell-related diseases. Specifically, disclosed is a use of a reagent which suppresses or activates proBDNF activity or proBDNF signaling pathways in preparing a regulator which regulates B lymphocyte function. Also disclosed is a use of a regulator in preparing a drug which treats B lymphocyte dysfunction-related diseases, a use of an antibody in preparing a reagent or reagent kit which detects/treats B lymphocyte dysfunction-related diseases, and a method for predicting a treatment or prevention effect of treating or preventing B lymphocyte dysfunction-related diseases.

### FIELD OF THE INVENTION

The present disclosure belongs to the field of treatment of B cell related diseases, and specifically relates to the use of proBDNF regulators in the diagnosis, prevention, treatment or improvement of B cell related diseases.

### BACKGROUND OF THE INVENTION

Brain derived neurotrophic factor (BDNF) is a neurotrophic factor discovered after nerve growth factor. It has a molecular weight of 12.4 kDa and is mainly distributed in the central nervous system. It has important functions in regulating survival, differentiation, plasticity of synapses and damage repair of neurons, and the like.

Precursor for brain-derived neurotrophic factor, ProBDNF, is synthesized in the endoplasmic reticulum after transcription and translation of BDNF gene. It is a peptide chain with 247 amino acids in length. Positions 1-18 of the amino acid sequence of ProBDNF are the signal peptide sequence. Two fragments are generated during the secretion process. One fragment is a polypeptide fragment comprising amino acids 19-129 of the sequence(i.e. the precursor domain), known as the proBDNF pro-domain, and the other fragment is positions 130-247 of the amino acid sequence(i.e. the fragment encoded by the mature domain), which is processed to form biologically active mBDNF.

At present, a mass of evidence shows that proBDNF is not only used as an intermediate product of mature BDNF synthesis, but also as a ligand which binds to its high affinity receptor p75 neurotrophin receptor (p75NTR, SEQ ID NO: 35), sortilin (SEQ ID NO: 36), SORCS2 (SEQ ID NO: 37) to play a biological role. ProBDNF binds to the extracellular domain of p75NTR and sortilin (SEQ ID NO: 38), forming a complex, and transducing several signals comprising RhoA, JNK and NFkB. There're researches showing that the precursors for neurotrophic factors (including proNGF and proBDNF, etc.) promote cell apoptosis and inflammatory response, but the role of proBDNF and its signals in B cell-related diseases remains unclear.

### SUMMARY OF THE INVENTION

### The Problem to be Solved by the Invention

Based on the existing problems in the prior art, it is necessary to provide a reagent capable of inhibiting or activating the activity of proBDNF or the proBDNF signaling pathway for the preparation of regulators for regulating the function of B lymphocytes. At the same time, there is also a need to provide an effective method for pre-detecting related diseases caused by B lymphocyte dysfunction, and an effective method for predicting the treatment or prevention effect of treating or preventing B lymphocyte dysfunction related diseases.

### Solutions to the Problem

In a technical solution, the present disclosure relates to the use of an agent for inhibiting or activating the activity of proBDNF or proBDNF signaling pathway in preparing a regulator for regulating B lymphocyte function, wherein the proBDNF signaling pathway is selected from the group consisting of p75NTR signaling pathway, sortinlin signaling pathway, or SORCS2 signaling pathway. In a technical solution, the sequence of proBDNF described in the present disclosure is the sequence shown in SEQ ID NO: 1, or a sequence having at least 80% identity with SEQ ID NO: 1; preferably, the sequence of proBDNF has at least 90% identity with SEQ ID NO: 1, more preferably, the sequence of proBDNF has at least 95% identity with SEQ ID NO: 1, and most preferably, the sequence of proBDNF has at least 99% identity with SEQ ID NO: 1.

In a technical solution, the use of the regulator of the present disclosure comprises one or more of the following uses:

(1) regulating B cell activation, proliferation and antigen presentation;
(2) reduceing antibody production;
(3) inhibiting plasmablast differentiation or cytokine production.

In a technical solution, the regulator described in the present disclosure is selected from the group consisting of proBDNF antagonist, p75NTR antagonist, Sortilin antagonist or SORCS2 antagonist.

In a technical solution, the use of the antagonist described in the present disclosure comprises one or more of the following uses:

(1) inhibiting or reducing the expression of proBDNF;
(2) interfering with, blocking or preventing the interaction or binding of proBDNF with p75NTR, sortilin or SORCS2;
(3) inhibiting or reducing the expression of p75NTR;
(4) interfering with, blocking or preventing the interaction or binding of p75NTR with proBDNF or sortilin.

In a technical solution, the proBDNF antagonist described in the present disclosure comprises an antibody or protein fragment, siRNA or small molecule compound against proBDNF.

In a technical solution, the p75NTR antagonist described in the present disclosure comprises an antibody against p75NTR, a p75NTR Fc fragment, a p75NTR ECD fragment and/or a small molecule compound.

In a technical solution, the proBDNF antagonist described in the present disclosure specifically recognizes the precursor domain of proBDNF.

In a technical solution, the sequence of the precursor domain of proBDNF described in the present disclosure has the sequence shown in SEQ ID NO: 2, or has at least 80% identity with SEQ ID NO: 2, preferably at least 90% identity, more preferably at least 95% identity, and most preferably at least 99% identity.

In a technical solution, the proBDNF antagonist described in the present disclosure specifically recognizes the sequence shown in SEQ ID NO: 27, 28 or 29, or specifically recognizes a sequence having at least 80% identity, preferably at least 90% identity, more preferably at least 95% identity, and most preferably at least 99% identity with the sequence shown in SEQ ID NO: 27, 28 or 29.

In a technical solution, the proBDNF antagonist described in the present disclosure is an antibody.

In a technical solution, the antibody described in the present disclosure is selected from the group consisting of Fab, Fab′, F(ab′)₂, Fv, single chain antibody, scFv, diabody, dsFv, single domain antibody, and/or peptide at least partially comprising a complementary determining region.

In a technical solution, the antibody described in the present disclosure is selected from the group consisting of:

antibody 1: HCDR1, HCDR2, and HCDR3 of the heavy chain have the sequences shown in SEQ ID NOs: 3, 4, and 5 respectively, or have the sequences shown in SEQ ID NOs: 13, 14, and 15 respectively;
antibody 2: LCDR1, LCDR2, and LCDR3 of the light chain have the sequences shown in SEQ ID NO: 6, 7, and 8 respectively, or have the sequences shown in SEQ ID NO: 16, 17, and 18 respectively;
antibody 3: having the heavy chain CDR region of antibody 1 and the light chain CDR region of antibody 2;
antibody 4: HCDR1, HCDR2 and HCDR3 of the heavy chain have the sequences shown in SEQ ID NOs: 3, 4 and 5 respectively, and LCDR1, LCDR2 and LCDR3 of the light chain have the sequences shown in SEQ ID NOs: 6, 7, and 8 respectively;
antibody 5: HCDR1, HCDR2 and HCDR3 of the heavy chain have the sequences shown in SEQ ID NOs: 13, 14, and 15 respectively, and LCDR1, LCDR2 and LCDR3 of the light chain have the sequences shown in SEQ ID NOs: 16, 17, and 18 respectively.

In a technical solution, the antibody has:

the heavy chain variable region shown in SEQ ID NO: 9 and the light chain variable region shown in SEQ ID NO: 10; or
the heavy chain variable region shown in SEQ ID NO: 19 and the light chain variable region shown in SEQ ID NO: 20.

In a technical solution, the antibody described in the present disclosure is:

an antibody with human immunoglobulin Fc region, formed by fusing the sequences of the heavy chain variable region shown in SEQ ID NO: 9 and the light chain variable region shown in SEQ ID NO: 10 with one or more heavy chain constant regions; or
an antibody with human immunoglobulin Fc region, formed by fusing the sequences of the heavy chain variable region shown in SEQ ID NO: 19 and the light chain variable region shown in SEQ ID NO: 20 with one or more heavy chain constant regions.

In a technical solution, the antibody described in the present disclosure is a monoclonal antibody, a murine antibody, a chimeric antibody, a humanized antibody, or a fully human antibody.

In another technical solution, the present disclosure also relates to the use of a regulator in the preparation of a medicament for treating B lymphocyte dysfunction-related diseases, wherein it treats the diseases by the following method, comprising administering to a subject a therapeutically effective amount of the regulators.

In another technical solution, the B lymphocyte dysfunction-related diseases are selected from the group consisting of B lymphocyte tumor, infectious disease, atherosclerosis, premature birth, body fluid rejection of transplant patients, graft-versus-host disease (GVHD) of transplant recipients, post-transplant lymphoproliferative disease.

In another technical solution, the B lymphocyte tumor is selected from the group consisting of chronic lymphocytic leukemia (CLL), small lymphocytic lymphoma (SLL), B cell prolymphocytic leukemia (B-PLL), non-CLL/SLL lymphoma, mantle cell lymphoma, multiple myeloma, Waldenstrom's macroglobulinemia, or a combination thereof.

In another technical solution, the B lymphocyte is selected from the group consisting of a circulating B lymphocyte, a blood B lymphocyte, a splenic B lymphocyte, a marginal zone B lymphocyte, a follicular B lymphocyte, a peritoneal B lymphocyte and/or a bone marrow B lymphocyte.

In another technical solution, when treating or improving the B lymphocyte tumor, the regulator and other anti-tumor drugs are administered simultaneously or sequentially.

In another technical solution, it prevents, treats, or improves the atherosclerosis, premature birth, body fluid rejection of transplant patients, and graft-versus-host disease (GVHD) of transplant recipients or post-transplant lymphoproliferative disease, while simultaneously administering the regulator and other immune antagonists.

In another technical solution, the present disclosure also relates to a method for predicting the therapeutic or preventive effect of treating or preventing B lymphocyte dysfunction-related diseases, comprising the following steps:

(1) detecting the expression level of proBDNF in a subject;
(2) administing the regulator according to any one of claims 1-16 to the subject;
(3) detecting the change in the expression level of proBDNF in the subject relative to (1) after the administration of the regulator shown in (2).

In another technical solution, the present disclosure also relates to the use of an antibody in the preparation of a reagent or kit for detecting/treating B lymphocyte dysfunction-related diseases, wherein the antibody is selected from the group consisting of proBDNF antibody, p75NTR antibody, sortinlin antibody or SORCS2 antibody.

### Effect of the Invention

In a technical solution, the present disclosure demonstrates the function of proBDNF in B cells, and for the first time proves that proBDNF regulators cause B cell activation, proliferation and antigen presentation, reduce antibody production, or inhibit plasmablast differentiation and cytokine production.

In a technical solution, the present disclosure provides a reagent that inhibits or activates the activity of proBDNF or the proBDNF signaling pathway. The aforementioned reagent comprises monoclonal antibodies that regulates B cell function and isuseful in treating B lymphocyte dysfunction-related diseases. The aforementioned proBDNF signaling pathway is selected from the group consisting of p75NTR signaling pathway, sortinlin signaling pathway, or SORCS2 signaling pathway. Wherein, the aforementioned monoclonal antibody is selected from the group consisting of 1D3 and 2H8, and has a good affinity for human proBDNF.

In a technical solution, the present disclosure provides a reagent or kit, which is useful in effective pre-detection of related diseases caused by B lymphocyte dysfunction.

In a technical solution, the present disclosure provides an effective method for predicting the therapeutic or preventive effects of treating or preventing B lymphocyte dysfunction-related diseases.

### BRIEF DESCRIPTION OF THE DRAWINGS

A and B in FIG. 1 show the kinetic curves of monoclonal antibodies 1D3 and 2H8, respectively, in the Biacore affinity determination experiment.

FIG. 2 shows the concentration of proBDNF in the supernatant after B cell activation.

FIGS. 3A and 3B show the the expression of proBDNF (A) and mBDNF (B) in PBMC cells under different conditions detected by using Elisa.

FIGS. 4A and 4B show the effects of different agonists on the expression of proBDNF (A) and mBDNF (B) in PBMCs detected by Western blot.

FIG. 5 shows the flow cytometric fluorescence intensity of CD40 and HLA-DR on the surface of CD19+B cells after CpGB treatment; wherein, the two columns of the IgG group and the two columns of the proBDNF group in A and B in FIG. 5 are the relative fluorescence intensity, the left column is normal saline, and the right column is CpGB.

A in FIG. 6 and B in FIG. 6 show the effect of CpGB treatment on B cell proliferation; C in FIG. 6 shows a flow cytometry graph of CSFE stained B cells.

FIG. 7 shows the effect of proBDNF antibody on CpGB-induced antibody production by B cells in in PBMCs.

FIG. 8 shows the effect of proBDNF antibody on CpGB promoting B cell differentiation.

FIG. 9 shows the ELISA results of 1D3 and 2H8 with polypeptides El-E5.

FIG. 10 shows the ELISA results of 1D3 and 2H8 with polypeptide DH.

FIG. 11 shows the fluorescence intensity of p75(CD271) in B cells stimulated by CpGB, Anti-IgM, PHA and LPS.

### DETAIL DESCRIPTION OF THE INVENTION

The present disclosure relates to the application of pro-BDNF regulators in B cell-related diseases. To facilitate the understanding of the present disclosure, some terms are defined as follows.

When used in conjunction with the term “comprising” in the claims and/or specification, the word “a” or “an” can mean “one”, but can also mean “one or more”, “at least one” and “one or more than one”.

As used in the claims and specification, the words “comprise”, “have”, “include” or “contain” mean inclusive or open-ended, and do not exclude additional, unquoted elements or method steps.

Although the disclosed content supports the definition of the term “or” only as an alternative and “and/or”, unless expressly stated that it's only an alternative or that the alternatives are mutually exclusive, the term “or” in the claims means “and/or”.

When used in the claims or specification, the selected/optional/preferred “numerical range” comprises both the value range endpoints at both ends of the range and all natural numbers covered between the numerical endpoints relative to the foregoing numerical endpoints. For example, the “sequence having at least 80% identity” described in the present disclosure refers to the sequence having 80%, 81%, 82%, 83%, 84%, 85%, 86%, 87%, 88%, 89%, 90%, 91%, 92%, 93%, 94%, 95%, 96%, 97%, 98%, 99%, 100% identity relative to the standard sequence.

The term “proBDNF” (precursor for brain-derived neurotrophic factor) is “precursor of brain-derived neurotrophic factor”, which is the precursor product of BDNF gene, and the sequence defined in SEQ ID NO: 1 is the nucleic acid sequence of human pro-BDNF. Positions 1-18 of the amino acid sequence of proBDNF are the signal peptide sequence. Two fragments are generated at this site during the secretion process, one of which is a polypeptide fragment comprising amino acids 19-129 (i.e. the precursor domain) of the sequence, which is refered to as proBDNF pro-domain, and the sequence defined in SEQ ID NO: 2 is the nucleic acid sequence of the precursor domain of human pro-BDNF; the other fragment is positions 130-247 of the amino acid sequence(i.e. the fragment encoded by the mature domain), which is processed to form mature BDNF with biological activity.

The term “p75NTR” is the neurotrophin receptor (P75 neurotrophin receptor, p75NTR), also known as CD271, which is the ligand of proBDNF, and may have the same meaning as P75 herein. p75NTR is a low-affinity receptor for neurotrophic factors, belonging to the members of the tumor necrosis factor receptor (TNFR) superfamily, and the earliest known NT (neurotrophin) receptor to be isolated. It is mainly expressed abundantly during the early development of nerve cells, and through different signaling pathways, it induces neurocyte dominated cell proliferation, migration, differentiation, survival, apoptosis, synapse establishment and nerve formation to exert multiple biological effects. At present, a large amount of evidence shows that proBDNF not only acts as an intermediate product of mature BDNF synthesis, but also acts as a ligand which binds to its high-affinity receptor p75NTR to exert biological effects.

The term “Sortilin” is a sorting protein, which belongs to the prototype members of the Vps10p-domain receptor family It can also be refered to as neurotensin receptor 3 (NTR3), glycoprotein 95 (GP95) or 100 kDa NT receptor. Sorting proteins are type I membrane receptors expressed in many tissues comprising brain, spinal cord, testis, liver and skeletal muscle (Petersen, Nielsen et al. 1997; Hermans-Borgmeyer, Hermey et al. 1999).

The term “SORCS2” stands for receptor for vacuolar protein sorting protein, another member of the receptor family of vacuolar protein sorting protein domain. SORCS2 has 1150 amino acids and is a type I transmembrane glycoprotein receptor. It is highly expressed in the brain and kidneys. proBDNF can combine with SORCS2 to form a proBDNF-p75NTR-SORCS2 complex.

The term “B lymphocyte”, or B cell for short, is a pluripotent stem cell derived from bone marrow. The differentiation process of mammalian B cells can be mainlydivided into five stages: pre-B cells, immature B cells, mature B cells, activated B cells and plasmocytes. The differentiation of pre-B cells and immature B cells is antigen independent, and the differentiation process is carried out in the bone marrow. The antigen-dependent stage refers to that mature B cells are activated after antigen stimulation and continue to differentiate into plasmocytes that synthesize and secrete antibodies. The differentiation at this stage is mainly carried out in the peripheral immune organs. Plasmocytes can synthesize and secrete antibodies and circulate in the blood. B-cell lymphoma is the most common lymphocytic leukemia. Stem cells or pre-B cells from the bone marrow, after immigrating into the bursa of fabricius or bursa of fabricius-like organs, gradually differentiate into B cells with immune potential. Mature B cells migrate out through the peripheral blood and enter the spleen and lymph nodes. They are mainly distributed in the spleen nodules, splenic cord and lymph nodules, lymphatic cords and submucosal lymph nodes of the digestive tract. After being stimulated by antigens, they differentiate and proliferate into plasmocytes, synthesizing antibodies to exert humoral immunity function. There are more B cells in bone marrow and collecting lymph nodes than T cells, less B cells in blood and lymph nodes than T cells, and even fewer in thoracic ducts, and only a few participate in recirculation. There are many different markers on the cell membrane of B cells, mainly surface antigens and surface receptors. These surface markers are giant protein molecules bound to the cell membrane. B1 cells are T cell independent cells. B2 is a T cell dependent cell. Survival time of B cells in the body is short, only a few days to a few weeks, but the memory cells of B cells can exist in the body for a long time. B lymphocyte dysfunction related diseases comprise B lymphocyte tumors, infectious diseases, atherosclerosis and premature birth.

The term “antagonist” refers to a class of substances that can bind to receptors but have no intrinsic activity. An antagonist is opposite to the term “agonist”. After binding to a receptor, an antagonist cannot induce a conformational change whichinduces biological activity change. The term “agonist” is capable of inducing a conformational change of the receptor to trigger biological activity. The term “p75NTR antagonist” as used in the present disclosure refers to a molecule that inhibits the expression level of components in the proBDNF-p75NTR ligand-receptor system in B cells (also referred to as “an expression antagonist”); or, that inhibits the interaction or binding between components of the proBDNF-p75NTR ligand-receptor system expressed in B cells (also called “a binding antagonist”), thereby reducing the amount, formation, and function of the ligand-receptor system, and/or its downstream signals.

The term “proBDNF antagonist” as used in the present disclosure can inhibit B cell activation, proliferation, antigen presentation, reduce antibody production, and/or inhibit plasmablast differentiation, cytokine production, etc. If a certain molecule causes a significant decrease in the expression of a component (transcription or translation level), it is considered that the molecule inhibits the expression level of the system component. Similarly, if certain molecules cause a significant reduction in the binding between the component and the formed ligand-receptor complex, it results in a significant reduction in downstream signals and functions mediated by the ligand-receptor system, such as reduction in antibody production, it is considered that the molecule inhibits the binding between components of the ligand-receptor system.

Binding antagonists can work in two ways. Binding antagonists can compete with the ligand proBDNF for receptors, thereby interfering with, blocking or preventing the binding of proBDNF to p75NTR, sortilin and/or SORCS2. This type of antagonist, which binds to the receptor without triggering the expected signal transduction, is also refered to as a “competitive antagonist”, which can comprise, for example, an oligopeptide designed based on the proBDNF sequence, or an antibody against SORCS2. Alternatively, the binding antagonist can bind to and isolate the ligand proBDNF, which has sufficient affinity and specificity with the ligand, and can substantially interfere with, block or prevent the binding of proBDNF to p75NTR, sortilin and/or SORCS2. This type of antagonist is also referred to as a “neutralizing antagonist”, which may comprise, for example, an antibody or aptamer directed against proBDNF, which specifically binds to proBDNF.

The characteristics of an antagonist can also be determined based on the target molecule to be antagonized by the antagonist. For example, a proBDNF antagonist refers to a molecule that inhibits or reduces the expression of proBDNF, or interferes with, blocks, or prevents the interaction or binding of proBDNF with p75NTR, sortilin, and/or SORCS2. On the other hand, a p75NTR antagonist refers to a molecule that inhibits or reduces the expression of p75NTR, or interferes with, blocks, or prevents the interaction of p75NTR with proBDNF and/or sortilin.

The term “protein fragment” refers to immunoglobulin molecules and immunoreactive parts of immune molecules, that is, molecules that comprise an antigen binding site which specifically binds to an antigen (“immune response”). The term “protein fragment” also comprises immunoglobulin molecules derived from various species, comprising invertebrates and vertebrates. Structurally, the simplest naturally occurring antibody (e.g., IgG) comprises four polypeptide chains: two heavy (H) chains and two light chain (L) chains interconnected by disulfide bonds Immunoglobulins represent a large family of molecules comprising several types of molecules, such as IgD, IgG, IgA, IgM, and IgE. The term “protein fragment” comprises, for example, hybrid antibodies or modified antibodies and fragments thereof. It has been shown that the antigen-binding function of antibodies can be performed by fragments of naturally occurring antibodies. These fragments are collectively referred to as “antigen binding units”. The term “protein fragment” also comprises any polypeptide chain containing molecular structuresthat have specific shapes that matches the epitopes and recognizes the epitopes, wherein one or more non-covalent binding interactions stabilize the complex between the molecular structure and the epitope. Examples of the antigen binding unit comprise Fab fragments, monovalent fragments consisted of VL, VH, CL and CH1 domains, and bivalent fragments comprising two Fab fragments connected by a disulfide bridge on the hinge region (F(ab)2 fragments); Fd fragments consisted of VH and CH1 domains, Fv fragments consisted of single-arm VL and VH domains of antibodies; dAb fragments consisted of VH domains (Ward et al., Nature, 341:544-546, 1989); and an isolated complementary determining region (CDR) or any fusion protein comprising such antigen binding units.

As used herein, the term “antibody” refers to the full-length or one or more fragments of an antibody that retains the ability to specifically bind to an antigen (e.g., a part of pro-BDNF), or other polypeptides that have similar antibody binding activity. Naturally occurring “antibodies” are glycoproteins comprising at least two heavy (H) chains and two light (L) chains connected by interchain disulfide bonds. Each heavy chain is consisted of a heavy chain variable region (abbreviated as VH herein) and a heavy chain constant region. The heavy chain constant region is consisted of three domains CH1, CH2 and CH3. Each light chain is consisted of a light chain variable region (abbreviated as VL herein) and a light chain constant region. The light chain constant region consists of a domain CL. The VH and VL regions can be further subdivided into regions of high variability, refered to as complementary determining regions (CDR), separated by more conservative regions called framework regions (ER). Each VH and VL is consisted of 3 CDRs and 4 FRs arranged in the following order: 1-R1, CDR1, FR2, CDR2, FR3, CDR3, FR4 from the amino terminal to the carboxy terminal. The variable regions of the heavy and light chains comprise binding domains that interact with antigens. The constant region of an antibody can mediate the binding of immunoglobulins to host tissues or factors (comprising various cells of the immune system (such as effector cells) and the first component (C1q) of the classical complement system).

It has been shown that the antigen-binding function of antibodies can be performed by fragments of full-length antibodies. An antibody fragment refers to a partial region of the aforementioned intact antibody (such as a monoclonal antibody or a polyclonal antibody), for example, Fab, Fab′, F(ab′)2, Fv (antibody variable region), single chain antibodies (H chain, L chain, V region of H chain, V region of L chain, etc.), scFv, diabody (i.e., scFv dimer), dsFv (disulfide stabilized V region), and peptides at least partially comprising a complementary determining regions (CDRs), etc.

The terms “complementary determining region” and “CDR” refer to the amino acid sequence that confer antigen specificity and binding affinity in the variable region of an antibody. Generally, there are 3 CDRs (HCDR1, HCDR2, HCDR3) in each heavy chain variable region and 3 CDRs (LCDR1, LCDR2, LCDR3) in the light chain variable region.

The amino acid sequence boundaries of a given CDR can be determined using any of many well-known plans, comprising those described by Kabat et al. (1991), “Sequences of Proteins of Immunological Interest,” 5th edition, Public Health Service, National Institutes of Health, Bethesda, MD (“Kabat” numbering plan), Al-Lazikani et al. (1997) JMB 273, 927-948 (“Chothia” numbering plan).

Although the two domains VL and VH of the Fv fragment are encoded by separate genes, they can be joined using recombination methods through synthetic linkers that allow them to be produced as a single chain protein in which the VL and VH regions are paired (scFv) (see, for example, Bird et al. 1988, Science 242:423-426; and Huston et al. 1988, Proc. Natl. Acad. Sci. 85:5879-5883). Such single chain antibodies are also intended to be comprised in the term “antigen-binding fragment” of antibodies. These antibody fragments are obtained using conventional techniques known to those of ordinary skill in the art, and the functions of the fragments can be screened in the same manner as used for intact antibodies.

The term “single domain antibody” cloned from camel-derived heavy chain antibody (HCAb), a single chain antibody consisting of only one heavy chain variable region. Its size is only 2.4×4 nm. It is the smallest fragment capable of binding antigen, refered to as a single domain antibody (VHH) or nanobody.

The term “epitope” means a protein determinant capable of specifically binding an antibody. Epitopes usually consist of chemically active surface groups of molecules such as amino acids or sugar side chains, and usually have specific structural characteristics and charge characteristics.

The term “fully human antibody” is intended to comprise antibodies having variable regions in which the framework and CDR regions are derived from sequences of human origin. In addition, if the antibody comprises a constant region, then the constant region is also derived from such human sequences, for example, human germline sequences, or mutated versions of antibodies of human germline sequences or common framework sequences from human framework sequences analysis (e.g., as described in Knappik et al., 2000, J Mol Biol 296: 57-86). The fully human antibodies of the present disclosure comprise amino acid residues not encoded by human sequences (e.g., mutations introduced by random or site-directed mutagenesis in vitro or by somatic mutation in vivo). However, as used herein, the term “fully human antibody” is not intended to comprise antibodies in which CDR sequences derived from the germline of another mammalian species, such as a mouse, have been grafted onto human framework sequences.

The term “recombinant human antibody” comprises all human antibodies prepared, expressed, produced or isolated by recombinant means. Such as antibodies isolated from transgenic or transchromosomic animals (such as mice) of human immunoglobulin genes or hybridomas prepared therefrom, antibodies isolated from host cells transformed to express human antibodies, e.g., from transfected tumors, antibodies isolated from recombinant combinatorial human antibody library, and antibodies prepared, expressed, produced, or isolated by any other means comprising splicing all or part of human immunoglobulin gene sequences with other DNA sequences. Such recombinant human antibodies have variable regions in which both the framework and CDR regions are derived from human germline immunoglobulin sequences. However, in certain embodiments, such recombinant human antibodies can be subjected to in vitro mutagenesis (or, when transgenic animals with human Ig sequences are used, somatic mutagenesis in vivo), thereby although the recombinant amino acid sequences in the VH and VL regions are derived from human germline VH and VL sequences and are related to the sequences, they may not naturally exist in the human antibody germline repertoire in vivo.

The term “isotype” refers to the type of antibodies provided by the heavy chain constant region gene (e.g., IgM, IgE, IgG, such as IgG1 or IgG4).

Since the antigen binding specificity of an antibody is mainly provided by the CDR1, 2 and 3 regions, the VH CDR1, 2 and 3 sequences can be “mixed and matched” with the VL CDR1, 2 and 3 sequences (i.e., CDRs from different antibodies can be mixed and matched, and each antibody comprising VH CDR 1, 2 and 3 and VL CDR 1, 2 and 3 is generated as an other anti-pro-BDNF binding molecule of the present disclosure). The pro-BDNF binding of such “mixed and matched” antibodies can be tested using the binding assays described above and in the examples or other conventional assays (e.g., ELISA). When mixing and matching VH CDR sequences, structurally similar CDR sequences should be used to replace the CDR1, CDR2 and/or CDR3 sequences from a specific VH sequence. Likewise, when mixing and matching VL CDR sequences, structurally similar CDR sequences should be used to replace the CDR1, CDR2, and/or CDR3 sequences from a specific VL sequence. It is obvious to those of ordinary skill in the art that the novel VH and VH can be generated by substituting one or more VH and/or VL CDR region sequences with structurally similar sequences from the CDR sequences of the monoclonal antibodies of the present disclosure shown herein.

As used herein, if the variable region chain or full-length chain of an antibody is obtained from a system using human germline immunoglobulin genes, the human antibody comprises products that are specific germline sequences or heavy or light chain variable regions or full-length heavy or light chains derived therefrom. Such systems comprise immunizing transgenic mice with human immunoglobulin genes with the target antigen, or screening a library of human immunoglobulin genes displayed on phage with the target antigen. Human antibodies that are “products of” or “derived from” human germline immunoglobulin sequences are identified by comparing the amino acid sequences of the human antibodies with the amino acid sequences of human germline immunoglobulins and selecting the human germline immunoglobulin sequences that are closest in sequence to the sequences of the human antibodies (i.e., the greatest percentage identity). Human antibodies that are “products of” or “derived from” a specific human germline immunoglobulin sequence may comprise amino acids that differ from the germline sequence due to, for example, the intentional introduction of naturally occurring somatic mutations or site-specific mutations. However, the selected human antibody usually has at least 90% identity with the amino acid sequence encoded by the human germline immunoglobulin gene in amino acid sequence, and comprises amino acid residues that identify human antibodies as human when compared with the amino acid sequences of germline immunoglobulins of other species (e.g., murine germline sequences). In certain cases, the human antibody has at least 60%, 70%, 80%, 90%, or at least 95% or even at least 96%, 97%, 98% or 99% identity in amino acid sequence with the amino acid sequence encoded by germline immunoglobulin genes. Generally, a human antibody derived from a particular human germline sequence will show a difference of no more than 10 amino acids from the amino acid sequence encoded by the human germline immunoglobulin gene. In certain cases, human antibodies show a difference of no more than 5 or even no more than 4, 3, 2, or 1 amino acid from the amino acid sequence encoded by the germline immunoglobulin gene.

### ANTIBODIES WITH CONSERVATIVE MODIFICATIONS

In certain embodiments, the antibody of the present disclosure has a heavy chain variable region comprising HCDR1, HCDR2, and HCDR3 sequences and a light chain variable region comprising LCDR1, LCDR2, and LCDR3 sequences, wherein one or more of these CDR sequences have designated amino acid sequences based on the antibody 1D3 or 2H8 described herein or the conservative modifications thereof, and wherein the antibody or protein retains the desired functional properties of the anti-pro-BDNF antibody of the present disclosure.

As used herein, the term “conservative sequence modification” is intended to mean an amino acid substitution in which an amino acid residue is replaced by an amino acid residue with a similar side chain. Families of amino acid residues with similar side chains have been defined in the art. These families comprise amino acids with basic side chains (for example, lysine, arginine, histidine), amino acids with acidic side chains (for example, aspartic acid, glutamic acid), and amino acids with uncharged polar side chains (for example, glycine, asparagine, glutamine, serine, threonine, tyrosine, cysteine, tryptophan), amino acids with non-polar side chains (for example, alanine, valine, leucine, isoleucine, proline, phenylalanine, methionine), amino acids with (3-branched side chains (for example, threonine, valine, isoleucine) and amino acids with aromatic side chains (for example, tyrosine, phenylalanine, tryptophan, histidine). Therefore, one or more amino acid residues in the CDR region of the antibody of the present disclosure can be replaced with other amino acid residues from the same side chain families, and the functional assay described herein can be used to test the retained function of the altered antibody.

Modifications can be introduced into the antibodies disclosed herein by standard techniques known in the art such as site-directed mutagenesis and PCR-mediated mutagenesis.

In addition, in addition to antibodies 1D3 and 2H8, the present disclosure also covers homologous antibodies or proteins that retain the ideal functional properties of 1D3 and 2H8 antibodies.

At the same time, the use of a “therapeutically effective amount” of the anti-pro-BDNF antibody or protein described in the present disclosure can lead to a reduction in the severity of disease symptoms, an increase in the frequency and duration of the asymptomatic period, or the prevention of damage or disability caused by the disease.

The composition of the present disclosure can be administered via one or more routes of administration using one or more of a variety of methods known in the art. As those of ordinary skill in the art will understand, the route and/or mode of administration will vary depending on the desired result. Routes of administration of antibodies of the present disclosure comprise intravenous, intramuscular, intradermal, intraperitoneal, subcutaneous, spinal, or other parenteral routes of administration, such as by injection or infusion. As used herein, the phrase “parenteral administration” means administration modes other than enteral and topical administration, usually by injection, comprising but not limited to intravenous, intramuscular, intraarterial, intrathecal, intrasaccular, intraorbital, intracardiac, intradermal, intraperitoneal, transtracheal, subcutaneous, subepidermal, intraarticular, subcapsular, subarachnoid, intraspine, epidural and intrasternal injections and infusions.

Alternatively, the antibodies or proteins of the present disclosure can be administered via non-parenteral routes, e.g., topical, epidermal or mucosal administration routes, such as intranasal, oral, vaginal, rectal, sublingual, or topical.

The antibodies or proteins of the present disclosure can be prepared with carriers that will protect the antibodies from rapid release, such as controlled release formulations, comprising implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods used to prepare such dosage forms have been patented or well known to those skilled in the art. See, for example, Sustained and Controlled Release Drug Delivery Systems, J. R. Robinson, Editor, Marcel Dekker Inc., New York, 1978.

The therapeutic composition can be administered by medical means known in the art. For example, in one embodiment, the therapeutic composition of the present disclosure can be administered by needle-free subcutaneous injection, such as those disclosed in U.S. Pat. Nos. 5,399,163; 5,383,851; 5,312,335, and the like.

Examples of well-known implants and modules that can be used in the present disclosure comprise: U.S. Pat. No. 4,487,603, which discloses an implantable micro-infusion pump for dispensing drugs at a controlled rate; U.S. Pat. No. 4,486,194, which discloses a therapeutic device for administering drugs through the skin; U.S. Pat. No. 4,447,233, which discloses a drug infusion pump for delivering drugs at a precise infusion rate; U.S. Pat. No. 4,447,224, which shows a variable flow implantable infusion device for continuous drug delivery; U.S. Pat. No. 4,439,196, which discloses an osmotic drug delivery system with multi-chamber compartments; U.S. Pat. No. 4,475,196, which discloses an osmotic drug delivery system. Many other implants, delivery systems, and modules of such type are known to those of ordinary skill in the art.

In certain embodiments, the antibodies or proteins of the present disclosure can be formulated to ensure proper distribution in the body. For example, the blood-brain barrier (BBB) excludes many highly hydrophilic compounds. To ensure that the therapeutic compounds of the present disclosure to pass the BBB (if necessary), they can be formulated in, for example, liposomes. For the method of manufacturing liposomes, see, for example, U.S. Pat. Nos. 4,522,811, 5,374,548 and 5,399,331. The liposome may comprise one or more moieties that are selectively transported to specific cells or organs, thereby enhancing targeted drug delivery (see, for example, V. V. Ranade 1989, J. Cline Pharmacol. 29:685). Exemplary targeting moieties comprise folate or biotin (see, for example, U.S. Pat. No. 5,416,016 belonging to Low et al.), mannosides (Umezawa et al. 1988, Biochem. Biophys. Res. Commun. 153:1038); antibodies (P. G. Bloeman et al. 1995, FEBS Lett. 357:140; M. Owais et al. 1995, Antimicrob. Agents Chernother. 39:180); Surfactant protein A receptor (Briscoe et al. 1995, Am. J. Physiol. 1233:134); p120 (Schreier et al. 1994, J. Biol. Chem. 269:9090); see also Keinanen and Laukkanen 1994, FEBSLett. 346:123; Killion and Fidler 1994, Immunomethods 4:273.

### USES AND METHODS OF THIS DISCLOSURE

The antibodies or proteins of the present disclosure have diagnostic and therapeutic functions in vitro and in vivo. For example, these molecules can be administered to cells in culture (e.g., in vitro or in vivo) or in a subject (e.g., in vivo) to treat, prevent, or diagnose B cell related disorders.

The method is particularly suitable for the treatment, prevention or diagnosis of B cell malignancies, atherosclerosis, premature birth, autoimmune disorders, body fluid rejection of transplant patients, and graft-versus-host disease (GVHD) of transplant recipients and post-transplant lymphoproliferative disease.

Specifically, B cell malignancies comprise chronic lymphocytic leukemia (CLL), small lymphocytic lymphoma (SLL), B cell prolymphocytic leukemia (B-PLL), non-CLL/SLL lymphoma, mantle cell Lymphoma, multiple myeloma, Waldenstrom's macroglobulinemia, or a combination thereof.

In addition, B cell related disorders can also comprise multiple sclerosis, asthma, arthritis, or psoriasis, diabetes, etc. Such diseases also comprise allergies and allergic conditions, hypersensitivity reactions, chronic obstructive pulmonary disease, cystic fibrosis, and organ or tissue transplant rejection.

In addition, when regulators targeting proBDNF are used to treat, prevent and improve B cell related diseases, regulators of proBDNF can also be administered as the only active ingredient or in combination with other drugs e.g., immunosuppressants, immunoregulators, or other cytotoxic or anticancer agents (for example, as an adjuvant or in combination with them), for example, to treat or prevent the aforementioned diseases. For example, the proBDNF regulator can be used in combination with the following drugs: DMARD, e.g., gold salt, sulfasalazine, antimalarial drugs, methotrexate, D-penicillamine, azathioprine, mycophenolic acid, tacrolimus, sirolimus, minocycline, Leflunomide, glucocorticoids; calcineurin inhibitors, e.g., cyclosporin A or FK 506; regulators of lymphocyte recycling, e.g., FTY720 and FTY720 analogs; mTOR inhibitors, e.g., rapamycin 40-O-(2-hydroxyethyl)-rapamycin, CCI779, ABT578, AP23573 or TAFA-93; ascomycins with immunosuppressive properties, e.g., ABT-281, ASM981, etc.; corticosteroids; cyclophosphamide; Azathioprine; Leflunomide; Mizoribine; Mycophenolate mofetil; 15-deoxyspergualin or its immunosuppressive homologs, analogs or derivatives; immunosuppressive monoclonal antibodies, e.g., monoclonal antibodies directed against leukocyte receptors, such as MHC, CD2, CD3, CD4, CD7, CD8, CD25, CD28, CD40, CD45, CD58, CD80, CD86 or their ligands; others immunoregulatory compounds, such as a recombinant binding molecule having at least a part of the extracellular domain of CTLA4 or its mutants, e.g. at least a part of the extracellular domain of CTLA4 or its mutants which linked to non-CTLA4 protein sequence, e.g., CTLA4Ig (e.g., designated as ATCC 68629) or its mutants, such as LEA29Y; adhesion molecule inhibitors (e.g., LFA-1 antagonists, ICAM-1 or -3 antagonists, VCAM-4 antagonists or VLA-4 antagonists); or chemotherapy agents (e.g., paclitaxel, gemcitabine, cisplatin, adriamycin or 5-fluorouracil; anti-TNF drugs, such as monoclonal antibodies against TNF (e.g. infliximab, adalimumab, CDP870), or receptor constructs against TNF-RI or TNF-RII (e.g. etanercept, PEG-TNF-RI); pro-inflammatory cytokine blockers, IL1 blockers, such as anakinra or IL1 traps, canakinumab, IL13 blockers, IL4 blockers, IL6 blockers, IL17 blockers (e.g. secukinumab, broadalumab, ixekizumab); chemokine blockers (e.g. inhibitors or activators of proteases such as metalloproteinases), anti-IL15 antibodies, anti-IL6 antibodies, anti-IL4 antibodies, anti-IL13 antibodies, anti-CD20 antibodies, NSAIDs, such as aspirin or anti-infective agents.

The present disclosure will be further explained below in conjunction with specific examples. It should be understood that these examples are only used to illustrate the present disclosure and not to limit the scope of the present disclosure. The experimental methods that do not specify specific conditions in the following examples are usually according to conventional conditions such as those described in J. Sambrook et al., Molecular Cloning Experiment Guide, 3rd Edition, Science Press, 2002, or according to the conditions suggested by the manufacturer.

### Example 1. SCREENING OF SINGLE CHAIN ANTIBODIES FOR THE HUMAN PROBDNF PRECURSOR

Phage display technology was used to screen human proBDNF precursor protein specific antibodies from a fully human natural antibody library. For this purpose, the conventional phage display method in the art was adopted to prepare a phage display library, and four rounds of directed screening were carried out for the biotin-labeled human proBDNF precursor recombinant protein. Conventional operations in the art was adopted in the preparation method and display method of the phage display library, as disclosed in PCT/CN2016/096292, Antibody Phage Display: methods and protocols (edited by Robert Aitken, 2009).

Ninety-six clones were selected from the screened clones, and their ability to bind human proBDNF precursor was analyzed by single phage ELISA (enzyme-linked immunosorbent assay). Combined with sequencing analysis, It was observed that single chain antibody 1D3 (see SEQ ID NO: 24 for nucleotide sequence, SEQ ID NO: 23 for amino acid sequence) and 2H8 (see SEQ ID NO: 26 for nucleotide sequence, see SEQ ID NO: 25 for amino acid sequence), have strong binding signals to human proBDNF precursor protein in ELISA experiment.

1D3 and 2H8 protein monoclonal antibodies were prepared, and recombinant monoclonal antibodies 1D3 and 2H8 were obtain after purification.

The affinity and kinetic parameters of the 1D3 and 2H8 monoclonal antibodies were measured by the capture method using the Biacore T200 system (purchased from GE). The operation is referred to the instruction. The adopted concentrations of human proBDNF precursor protein were 0.25nM, 0.5nM, 1nM, 2nM, 4nM and 8nM, respectively. The Biacore T200 evaluation software was used to evaluate the obtained action curve and to calculate the KD value of affinity . FIG. 1A and FIG. 1B respectively show the kinetic curves of monoclonal antibodies 1D3 and 2H8 in the Biacore affinity determination experiment.

The binding data of 1D3 and 2H8 monoclonal antibodies to human proBDNF precursor protein were summarized in Table 1.

TABLE 1
Affinity parameters of monoclonal antibodies of
1D3 and 2H8 to human proBDNF precursor protein

| Antibody | | | |
|---|---|---|---|
| sample | ka (1/Ms) | kd (1/s) | KD (M) |
| 1D3 | 5.256E+06 | 7.994E−04 | 1.521E−10 |
| Monoclonal | | | |
| antibody | | | |
| 2H8 | 3.103E+06 | 5.940E−03 | 1.914E−09 |
| Monoclonal | | | |
| antibody | | | |

### EXAMPLE 2. SECRETION OF PROBDNF IN PBMCS STIMULATED UNDER DIFFERENT CONDITIONS

PBMCs were derived from the buffy coat of the peripheral blood of normal blood donors. PBMCs were isolated and counted according to conventional techniques in the art. The cells were diluted with complete RPMI medium to 2*10⁶/ml, and 250 ul cell suspension (500,000 cells per well) was added to each well of a 96-well plate, and cultured in a constant temperature incubator at 37° C. and 5% CO₂ for 72 h after adding different stimuli. The grouping and stimulus usage were as follows:

Normal control group (NT): no stimulus was added;
CpGB group (B cell agonist): 2.5 ul 100×CpG ODN B (Genework) was add, the working concentration was 3.2 ug/ml
Anti-IgM group (B cell agonist): 2.5 ul 100×anti-human IgM BCR cross-link antibody (Jackson ImmunoResearch) was add, the working concentration was 10 ug/ml;
Anti-CD3 group (T cell activator): lul Dynabeads® Human T-Activator CD3/CD28 (Thermol fisher scientific) was add, the working concentration of magnetic beads: cells=1:1;
Phytohemagglutinin (PHA, T cell activator): 2.5 ul 100×Phytohemagglutinin (PHA, Sigma) was add, the working concentration was 10 ug/ml. The ELISA detection of proBDNF was performed by the operation known in the art (such as J Neurochem. 2015), and the Graphpad prism 6.0 software was adopted for the analysis, and the measurement data was shown as mean±standard error. One-way analysis of variance was adopted for intergroup analysis, and pairwise comparisons within groups were analyzed by adopting Tukey or Bonferroni method. P<0.05 was considered as statistically significant.

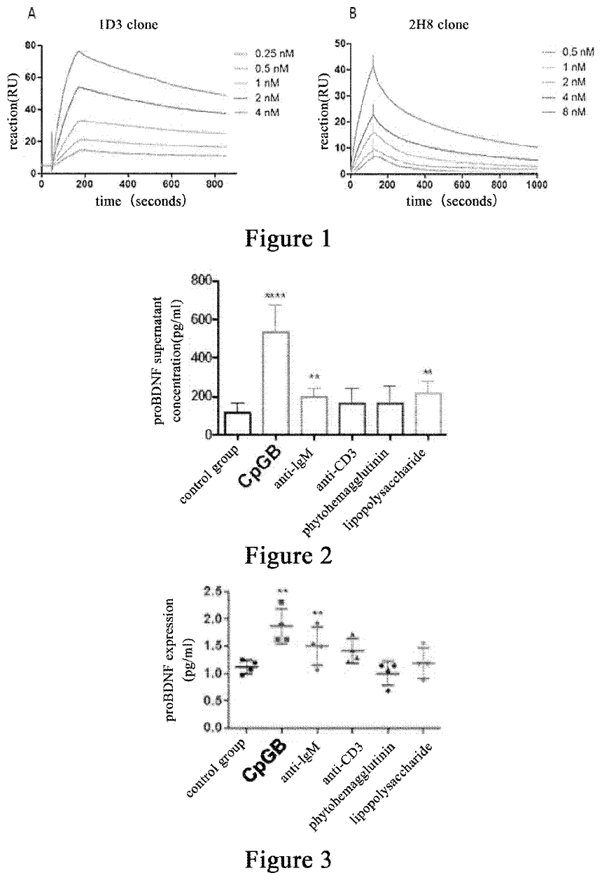

The results showed (FIG. 2): The release of proBDNF was significantly increased after stimulation by CpGB and anti-IgM; while the effect of anti-CD3 and PHA on the release of proBDNF was not obvious. These results indicated that B cell activators (CpGB and Anti-IgM) could promote PBMCs to secrete proBDNF. The T cell activator had no such effect, indicating that after activation, B cell caused the secretion of proBDNF.

### EXAMPLE 3. PROBDNF EXPRESSION OF PBMCS BY STIMULATION UNDER DIFFERENT CONDITIONS

The isolation and culturing of PBMCs was referred to Example 2, and the ELISA analysis is refer to the operation of Example 2 . The results showed (FIGS. 3A and 3B) that the expression of proBDNF in PBMCs was significantly up-regulated under CpGB and Anti-IgM stimulation (FIG. 3A); after CpGB, Anti-IgM, and PHA treatment, mBDNF in PBMCs was significantly down-regulated (FIG. 3B), suggesting B Cell agonists can increase the expression of proBDNF in PBMCs.

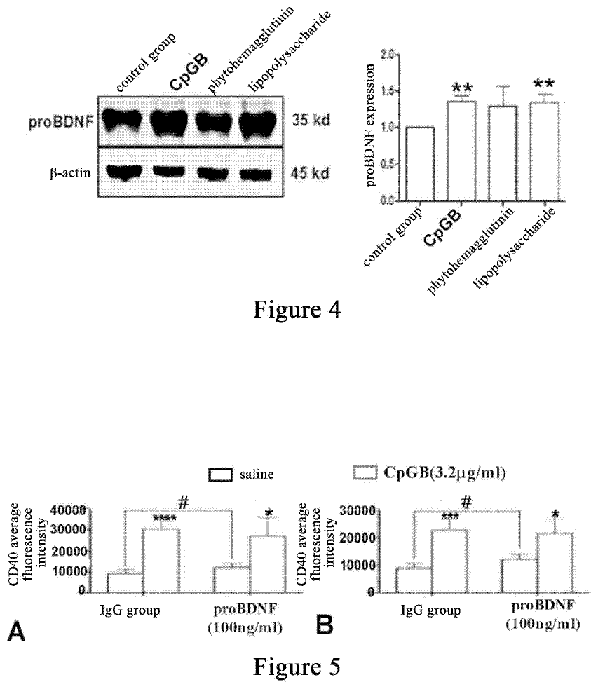

Western Blot analysis was adopted, and conventional technical means by those skilled in the art were adopted in the analysis method. The cell lysate was obtained by conventional operations in the art, and then the corresponding volume of loading buffer (Loading Buffer, Bio-RAD, Lot#161-0747) and 1/20 1M DTT were added to each tube at a ratio of 1:3, and heated in a heater at 98° C. for 10 minutes, and after cooling, 1/20 1M DTT was added again and then the lysate was subjected to Western Blot analysis. Anti-proBDNF primary antibody: fully human monoclonal antibody 1D3 was adopted, the working concentration was 1:1000; anti-mBDNF primary antibody: santa cruz, sc-546; the working concentration was 1:1000. The results were shown in FIG. 4, CpGB and PHA caused the up-regulation of proBDNF expression in PMBCs.

Both ELISA and Western Blot methods proved that activating B cells can lead to the up-regulation of proBDNF expression in PBMCs, indicating that B cells were the main source of proBDNF expression and secretion.

### EXAMPLE 4. THE EFFECT OF PROBDNF ON B CELL ACTIVATION

After counting PBMCs, the cells were diluted with complete RPMI medium to 2*10⁶/ml, and 250 ul cell suspension (500,000 cells per well) was added to each well of a 96-well plate, and cultured in a constant temperature incubator at 37° C. and 5% CO₂ for 72 hours after adding different stimuli. The grouping and stimulus usage were as follows:

Normal control group (NT): no stimulus was added;
proBDNF group: 2.5 ul 100×recombinant proBDNF protein was added, the working concentration was 100 ng/ml;
CpGB group: 2.5 ul 100×CpG ODN B (Genework) was added, the working concentration was 3.2 ug/ml;
CpGB+proBDNF group: CpGB and proBDNF stimuli were added simultaneously. Cell surface molecules were analysis by flow cytometry. The specific steps were the same as in Example 11. The combinations of fluorescence and antibodies on cell surface were as follows: CD19: FITC⁺; CD40: PE-Cy5⁺; HLA-DR: BV510.

The experimental results were shown in FIG. 5. After CpGB treatment, the flow fluorescence intensity of CD19⁺B cell surface molecules, CD40 and HLA-DR, were significantly higher than those of the untreated group. Exogenous proBDNF protein treatment (proB) promoteed the activation of resting B cells and up-regulateed the expression of CD40 and HLA-DR on the surface. These results indicate that proBDNF can activate B cells.

### EXAMPLE 5. EFFECT OF CLONE 1D3 ON THE PROLIFERATION OF B CELLS AND T CELLS IN PBMCS

After counting PBMCs, the cells were diluted with complete RPMI medium to 2*10⁶/ml, and 250 ul cell suspension (500,000 cells per well) was added to each well of a 96-well plate after staining the cells with CFSE, and cultured in a constant temperature incubator at 37° C. and 5% CO₂ for 72 hours after adding different stimuli. The grouping and stimulus usage were as follows:

Normal control group (NT): no stimulus was added;
proBDNF group: 2.5 ul 100×recombinant proBDNF protein was added every other day, working concentration was 50 ng/ml;
Ab-proBDNF group: 2.5 ul 100×Ab-proBDNF (1D3) was added, working concentration was 2 ug/ml;
CpGB group: 2.5 ul 100×CpG ODN B (Genework) was added, working concentration was 3.2 ug/ml; cells=1:1;
CpGB+proBDNF group: CpGB and proBDNF stimuli were added simultaneously;
CpGB+Ab-proBDNF group: CpGB and 1D3 stimuli were added simultaneously;
IgG isotype control group: CpGB/anti-CD3 and IgG isotype control (eBioscience, 2 ug/ml) were added respectively.

The CSFE method was adopted to detect the proliferation of B cells. Flow cytometry was used for the analysis of cell surface molecules. The combination of fluorescence and antibody on cell surface was as follows: CD19: FITC⁺.

The statistical results were shown in FIGS. 6A and 6B, and FIG. 6C shows the flow cytometry of CSFB stained B cells; the effect of CpGB treatment on B cell proliferation. Compared with the untreated group (NT group), CD19⁺B cells proliferated significantly after CpGB treatment. Compared with the CpGB group, the B cell proliferation of the CpGB+proBDNF group was significantly increased; the CpGB+Ab-proBDNF (1D3) group had a significant decrease in the degree of B cell proliferation compared with the CpGB group. These results indicate that the clone 1D3 monoclonal antibody can significantly inhibit the proliferation of B cells caused by CpGB treatment.

These results indicate that the clone 1D3 significantly inhibits the proliferation of B cells.

### EXAMPLE 6. EFFECT OF CLONE 1D3 ON CPGB-INDUCED ANTIBODY PRODUCTION BY B CELLS IN PBMCS

After counting PBMCs, the cells were diluted with complete RPMI medium to 2*10⁶/ml, and 250 ul cell suspension (500,000 cells per well) was added to each well of a 96-well plate, and cultured in a constant temperature incubator at 37° C. and 5% CO₂ for 10 days after adding different stimuli. The grouping and stimulus usage were as follows:

Normal control group (NT): no stimulus was added;
proBDNF group: 2.5 ul 100×recombinant proBDNF protein was added every other day, working concentration was 50 ng/ml;
Ab-proBDNF group: 2.5 ul 100×Ab-proBDNF (clone 1D3) was added, working concentration was 2 ug/ml;
CpGB group: 2.5 ul 100×CpG ODN B (Genework) was added, working concentration was 3.2 ug/ml;
CpGB+proBDNF group: CpGB and proBDNF stimuli were added simultaneously;
CpGB+Ab-proBDNF group: CpGB and Ab-proBDNF (clone 1D3) stimuli were added simultaneously;
IgG isotype control group: CpGB/anti-CD3 and IgG isotype control (eBioscience, 2 ug/ml) were added respectively.

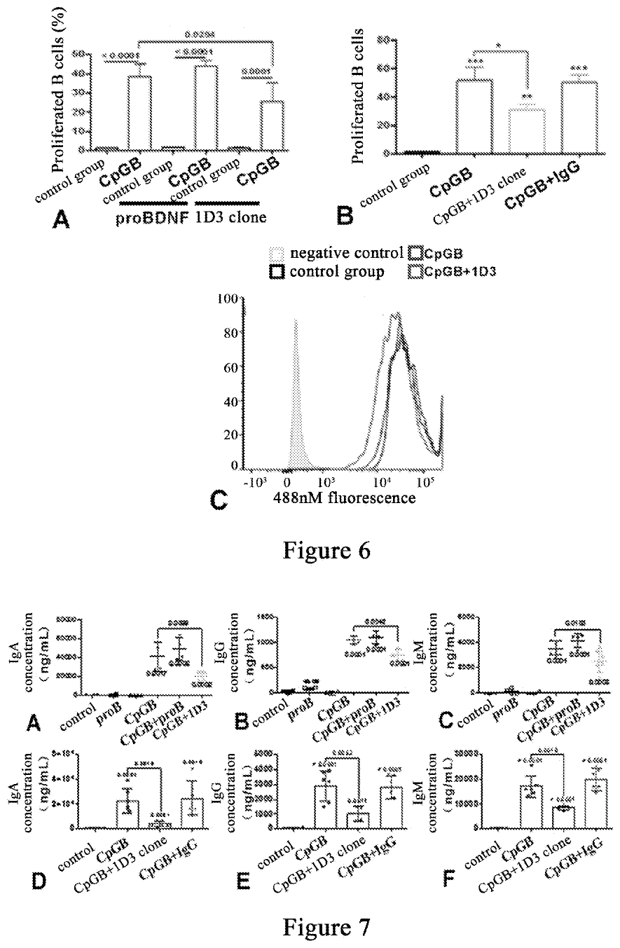

The supernatant of PBMCs was taken for the ELISA detection and analysis of IgA/IgG/IgM, and Prism6 was adopted to analyze the changes in the corresponding proBDNF and mBDNF protein content. The experimental results showed (FIG. 7) the protein content of IgA, IgG and IgM in the culture supernatant of PBMCs after CpGB stimulation was significantly higher than that of the unstimulated group (FIG. 7, red). The intervention of exogenous proBDNF slightly promoted the secretion of IgG in B cells (FIG. 7B). Ab-proBDNF (1D3) significantly inhibited the release of IgA, IgG and IgM induced by CpGB, which was manifested as a significant decrease in the secretion of these antibodies in the supernatant (FIG. 7, green).

These results indicate that clone 1D3 Ab-proBDNF significantly inhibits the secretion of various antibodies after B cell activation, and further illustrate that Ab-proBDNF inhibits the function of B cells to secrete antibodies.

### EXAMPLE 7. THE EFFECT OF CLONE 1D3 ON CPGB-PROMOTED DIFFERENTIATION OF B CELLS INTO CD27⁺CD38⁺ PLASMABLASTS

After counting PBMCs, the cells were diluted with complete RPMI medium to 2*10⁶/ml, and 250 ul cell suspension (500,000 cells per well) was added to each well of a 96-well plate, and cultured in a constant temperature incubator at 37° C. and 5% CO₂ for 10 days after adding different stimuli. The experiment was grouped as follows:

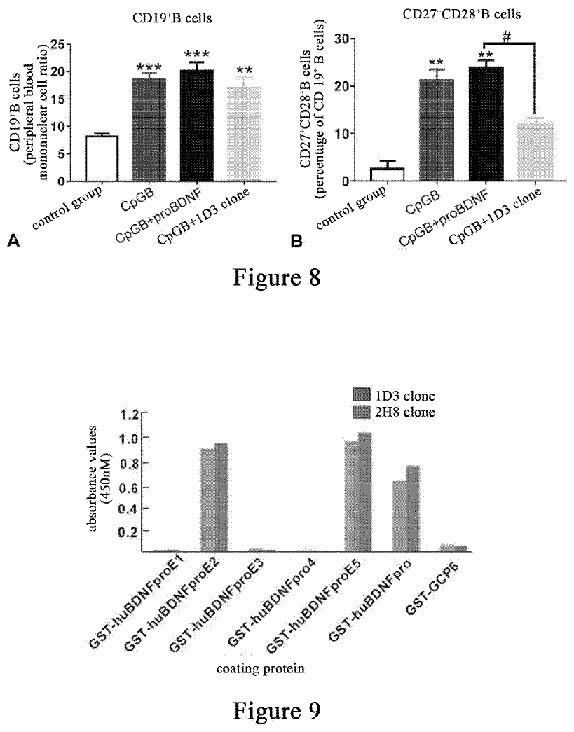

Normal control group (NT): no stimulus was added;
CpGB group: 2.5 ul 100×CpG ODN B (Genework) was add, working concentration was 3.2 ug/ml;
CpGB+proBDNF group: CpGB and proBDNF stimuli were added simultaneously;
CpGB+Ab-proBDNF group: CpGB and Ab-proBDNF (clone 1D3) stimuli were added simultaneously;
Cell surface molecules were analysis by flow cytometry, combinations of fluorescence and antibodies on cell surface were as follows: CD19: FITC+; CD27: BV421+; CD38: PE-CyS. The experimental results were shown in FIG. 8, wherein the percentage of the number of CD27⁺CD38⁺ plasmablasts (relative to the total number of CD19⁺B cells) increased significantly, indicating that CpGB can promote the differentiation of B cells into plasmablasts. Exogenous proBDNF slightly increased the number of CD19⁺B cells and CD27⁺CD38⁺ plasmablasts induced by CpGB treatment, but it was not statistically significant. In the CpGB+Ab-proBDNF group, the percentage of CD27⁺CD38⁺ plasmablasts in CD19+B cells was significantly lower than that in the CpGB group or CpGB+proBDNF group.

These results indicate that the clone 1D3 Ab-proBDNF antibody can inhibit the differentiation of B cells and plasmablasts, further indicating that the clone 1D3 Ab-proBDNF regulates B cell functions at multiple levels.

### EXAMPLE 8. EPITOPE BINDING EXPERIMENT OF ANTIBODY

The results of ELISA showed that antibodies 1D3 and 2H8 binded to denatured human proBDNF, suggesting that the binding epitopes of the two antibodies were likely to be linear. Human proBDNF was equally divided into two peptides (El, E2) or three peptides (E3, E4, E5), the amino acid sequence was as follows:

E1:
(SEQ ID NO: 30)
pmkeanirgqgglaypgvrthgtlesvngpkagsrgltsladtfehmiee
lldedq
E2:
(SEQ ID NO: 27)
kvrpneennkdadlytsrvmlssqvpleppllflleeyknyldaanmsmr
vrrh
E3:
(SEQ ID NO: 31)
pmkeanirgqgglaypgvrthgtlesvngpkagsrgl
E4:
(SEQ ID NO: 32)
tsladtfehmieelldedqkvrpneennkdadlytsr
E5:
(SEQ ID NO: 28)
vmlssqvpleppllflleeyknyldaanmsmrvrrh

The five peptides were fused with GST and expressed, and then purified. The binding of antibodies 1D3 and 2H8 was detected by ELISA. The results were shown in FIG. 9. Antibodies 1D3 and 2H8 bind all of full length GST-human proBDNF, GST-human proBDNF-E2 peptide, and GST-human proBDNF-E5 peptide, suggesting that the binding epitopes of these two antibodies were located within these 36 amino acids of the E5 peptide.

### EXAMPLE 9. EPITOPE BINDING EXPERIMENT OF ANTIBODY

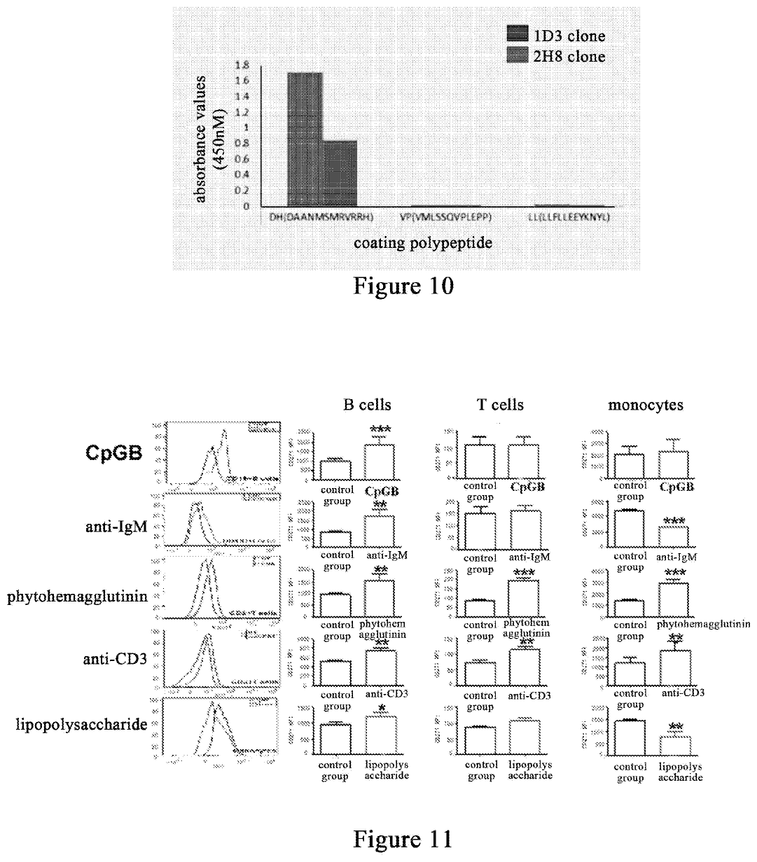

The E5 peptide was further divided into three equal peptide segments, each comprising 12 amino acids, namely VP peptide (VMLSSQVPLEPP, SEQ ID NO: 33), LL peptide (LLFLLEEYKNYL, SEQ ID NO: 34), DH Peptide (DAANMSMRVRRH, SEQ ID NO: 35). Biotinylated VP peptide, LL peptide and DH peptide were synthesized in vitro by GL Biochemical (Shanghai) Ltd. and the binding of antibodies was detected by ELISA. The results were shown in FIG. 10. The binding of antibody 1D3 to DH peptide was strong, the binding of 2H8 to DH peptide was weak, and there's no binding to the other two peptides, suggesting that the epitope of antibody 1D3 was in the DH peptide, and the epitope of 2H8 was near the DH peptide.

### EXAMPLE 10. EXPRESSION AND RELEASE OF P75NTR IN PBMCS (THE LIGAND WAS EXPRESSED ON THE SURFACE OF B CELLS)

PBMCs were isolated from peripheral blood, and cell surface molecules were analyzed by flow cytometry (BD FACSCanto II). The operation was refered to the product instruction. In addition to the samples to be tested, various control groups were set up. The treatments of each group were as follows:

Blank control group: 100 μl of PBS comprising 5% normal human serum was added; Isotype control group: 100 μl of PBS comprising 5 μl IgG isotype control (BD Pharmingen, Lot #555748) and 5% normal human serum was added;
Fluorescence compensation control group: 100 μl PBS comprising only one of the antibodies (CD3:PE-cy7 or CD19: FITC or CD14: APC-Cy7 or CD271: PE) and 5% normal human serum was added to each tube;
Group of samples to be tested: 100 pl of PBS comprising cell surface antibody mixture comprising various fluorescent color combinations (CD3:PE-cy7+CD19: FITC+CD14: APC-Cy7+CD271: PE) and 5% normal human serum was added;
The experimental results were shown in FIG. 11: the fluorescence intensity of p75(CD271) of was significantly higher than that of the normal non-stimulated control group when B cells were stimulated by CpGB, Anti-IgM, PHA and LPS; p75 on the cell surface was significantly up-regulated when T cells were stimulated by anti-CD3 or PHA, but did not change significantly under other stimuli. These results indicate that different agonists can cause up-regulation of p75 expression on the surface of B cells after activating PBMCs, suggesting that B cells is an important target of proBDNF.

The above-mentioned examples of the present disclosure are merely examples to clearly illustrate the present disclosure, and are not intended to limit the embodiments of the present disclosure. For those of ordinary skill in the art, other changes or modifications in different forms can be made on the basis of the above description. It is unnecessary and impossible to list all the embodiments here. Any modification, equivalent replacement and improvement made within the spirit and principle of this disclosure shall be comprised in the protection scope of the claims of this disclosure.

1. Use of an agent A method for inhibiting or activating the activity of proBDNF or proBDNF signaling pathway in preparing a regulator for regulating B lymphocyte function, wherein the proBDNF signaling pathway is selected from the group consisting of p75NTR signaling pathway, sortinlin signaling pathway, or SORCS2 signaling pathway.

2. The method according to claim 1, wherein the amino acid sequence of proBDNF is the sequence which is encoded by the sequence shown in SEQ ID NO: 1, or a amino acid sequence having at least 80% identity with the sequence which is encoded by the sequence shown in SEQ ID NO: 1; preferably, the amino acid sequence of proBDNF has at least 90% identity with the sequence which is encoded by the sequence shown in SEQ ID NO: 1, more preferably, the amino acid sequence of proBDNF has at least 95% identity with the sequence which is encoded by the sequence shown in SEQ ID NO: 1, and most preferably, the amino acid sequence of proBDNF has at least 99% identity with the sequence which is encoded by the sequence shown in SEQ ID NO: 1.

3. The method according to claim 1, wherein the regulator comprises:
(1) regulates B cell activation, proliferation and antigen presentation;
(2) reduces antibody production; and/or
(3) inhibits plasmablast differentiation or cytokine production.

4. The method according to claim 1, wherein the regulator is selected from the group consisting of a proBDNF antagonist, p75NTR antagonist, Sortilin antagonist and SORCS2 antagonist; and wherein the antagonist:
(1) inhibits or reduces the expression of proBDNF;
(2) interferes with, blocks, or prevents the interaction or binding of proBDNF with p75NTR, sortilin or SORCS2;
(3) inhibits or reduces the expression of p75NTR; and/or
(4) interferes with, blocks, or prevents the interaction or binding of p75NTR with proBDNF or sortilin.

5. (canceled)

6. The method according to claim 4, wherein the regulator is:
(a) a proBDNF antagonist comprising an antibody or protein fragment, siRNA or small molecule compound against proBDNF; or
(b) a p75NTR antagonist comprising an antibody against p75NTR, a p75NTR Fc fragment, a p75NTR ECD fragment and/or a small molecule compound.

7. (canceled)

8. The method according to claim 4, wherein the proBDNF antagonist specifically recognizes the precursor domain of proBDNF.

9. The method according to claim 8, wherein the sequence of the precursor domain of proBDNF has the sequence shown in SEQ ID NO: 2, or has at least 80% identity with SEQ ID NO: 2, preferably at least 90% identity, more preferably at least 95% identity, and most preferably at least 99% identity.

10. The method according to claim 8, wherein the proBDNF antagonist specifically recognizes the sequence shown in SEQ ID NO: 27, 28 or 29, or specifically recognizes a sequence having at least 80% identity, preferably at least 90% identity, more preferably at least 95% identity, and most preferably at least 99% identity with the sequence shown in SEQ ID NO: 27, 28 or 29.

11. The method according to claim 10, wherein the proBDNF antagonist is an antibody.

12. The method according to claim 11, wherein the antibody is selected from the group consisting of Fab, Fab′, F(ab′)2, Fv, single chain antibody, scFv, diabody, dsFv, single domain antibody, and/or peptide at least partially comprising a complementary determining region.

13. The method according to claim 11, wherein the antibody comprises:
(a) HCDR1, HCDR2, and HCDR3 of the heavy chain comprising the sequences shown in SEQ ID NOs: 3, 4, and 5 respectively, or the sequences shown in SEQ ID NOs: 13, 14, and 15 respectively;
(b) LCDR1, LCDR2, and LCDR3 of the light chain comprising the sequences shown in SEQ ID NO: 6, 7, and 8 respectively, or the sequences shown in SEQ ID NO: 16, 17, and 18 respectively;
(c) HCDR1, HCDR2 and HCDR3 of the heavy chain comprising the sequences shown in SEQ ID NOs: 3, 4 and 5 respectively, and LCDR1, LCDR2 and LCDR3 of the light chain comprising the sequences shown in SEQ ID NOs: 6, 7, and 8 respectively;
(d) HCDR1, HCDR2 and HCDR3 of the heavy chain comprising the sequences shown in SEQ ID NOs: 13, 14, and 15 respectively, and LCDR1, LCDR2 and LCDR3 of the light chain comprising the sequences shown in SEQ ID NOs: 16, 17, and 18 respectively;
(e) a heavy chain variable region comprising SEQ ID NO: 9 and a light chain variable region comprising SEQ ID NO: 10;
(f) a heavy chain variable region shown in SEQ ID NO: 19 and a light chain variable region shown in SEQ ID NO: 20;
(g) an antibody comprising a human immunoglobulin Fc region, formed by fusing the sequences of the heavy chain variable region shown in SEQ ID NO: 9 and the light chain variable region shown in SEQ ID NO: 10 with one or more heavy chain constant regions; or
(h) an antibody comprising a human immunoglobulin Fc region, formed by fusing the sequences of the heavy chain variable region shown in SEQ ID NO: 19 and the light chain variable region shown in SEQ ID NO: 20 with one or more heavy chain constant regions.

14. (canceled)

15. (canceled)

16. The method according to claim 13, wherein the antibody is a monoclonal antibody, a murine antibody, a chimeric antibody, a humanized antibody, or a fully human antibody.

17. A method of treating a B lymphocyte dysfunction-related disease in a subject, wherein the method comprises administering to the subject a therapeutically effective amount of a regulator selected from the group consisting of a proBDNF antagonist, p75NTR antagonist, Sortilin antagonist and SORCS2 antagonist.

18. The method according to claim 17, wherein the B lymphocyte dysfunction-related disease is selected from the group consisting of B lymphocyte tumor, infectious disease, atherosclerosis, premature birth, body fluid rejection of transplant patients, graft-versus-host disease (GVHD) of transplant recipients, and post-transplant lymphoproliferative disease.

19. The method according to claim 18, wherein the B lymphocyte tumor is selected from the group consisting of chronic lymphocytic leukemia (CLL), small lymphocytic lymphoma (SLL), B cell prolymphocytic leukemia (B-PLL), non-CLL/SLL lymphoma, mantle cell lymphoma, multiple myeloma, and Waldenstrom's macroglobulinemia, or a combination thereof.

20. The method according to claim 17, wherein the B lymphocyte is selected from the group consisting of a circulating B lymphocyte, a blood B lymphocyte, a splenic B lymphocyte, a marginal zone B lymphocytes, a follicular B lymphocyte, a peritoneal B lymphocytes and/or a bone marrow B lymphocyte.

21. The method according to claim 17 further comprising administering one or more additional immune antagonists and/or anti-tumor drugs either simultaneously or sequentially.

22. (canceled)

23. A method for predicting the therapeutic or preventive effect of treating or preventing B lymphocyte dysfunction-related diseases, comprising the following steps:
(1) detecting the expression level of proBDNF in a subject;
(2) administering a regulator selected from the group consisting of a proBDNF antagonist, p75NTR antagonist, Sortilin antagonist and SORCS2 antagonist to the subject;
(3) detecting the change in the expression level of proBDNF in the subject relative to (1) after the administration of the regulator shown in (2).

24. A kit for detecting/treating a B lymphocyte dysfunction-related disease in a subject comprising:
(a) an antibody is selected from the group consisting of proBDNF antibody, p75NTR antibody, sortinlin antibody and SORCS2 antibody; and
(b) instructions for use.
